(19) United States
(12) Patent Application Publication
(10) Pub. No.: US 2009/0157152 A1
(43) Pub. Date: Jun. 18, 2009

(54) COSMETIC METHOD FOR IMPROVING SKIN CONDITION OF FACE AND NECK, AND APPARATUS THEREOF

(75) Inventor: Tomonobu Ezure, Yokohama-shi (JP)

Correspondence Address: DARBY & DARBY P.C., P.O. BOX 770, Church Street Station, New York, NY, 10008-0770, US

(73) Assignee: SHISEIDO COMPANY, LTD., Tokyo (JP)

(21) Appl. No.: 12/253,758

(22) Filed: Oct. 17, 2008

Related U.S. Application Data
(60) Provisional application No. 60/981,181, filed on Oct. 19, 2007.

(30) Foreign Application Priority Data
Aug. 29, 2008 (JP) 2008-222434
Oct. 15, 2008 (JP) 2008-265937

(51) Int. Cl. A61F 7/00; H05B 1/00

(52) U.S. Cl. 607/109; 219/201

(57) ABSTRACT

It is an object of the invention to develop a method and an apparatus for improving a skin condition by improving the condition of the subcutaneous fat tissue that induces abnormal secretion of adipocytokines by the subcutaneous fat cells. The present invention provides a method for improving skin condition of the face and/or neck. The method for improving skin condition of the present invention comprises a step for applying 60 to 90 minutes of thermal stimulation at 41 to 43° C. to the subcutaneous fat of the face and/or neck. The present invention also provides an apparatus for improving the skin condition of the face and/or neck. The apparatus for improving skin condition of the present invention comprises a heater and applies 60 to 90 minutes of thermal stimulation at 41 to 43° C. to the subcutaneous fat of the face and/or neck.

### CROSS REFERENCE TO RELATED APPLICATIONS

The present application is based on and claims priorities of U.S. provisional patent application No. 60/981,181 filed on Oct. 19, 2007; Japanese patent application No. 2008-222434 filed on Aug. 29, 2008; and Japanese patent application No. 2008-265937 filed on Oct. 15, 2008, the entire contents of which are hereby incorporated by reference.

### BACKGROUND OF THE INVENTION

1. Field of the Invention

The present invention relates to a cosmetic method and an apparatus for improving the skin condition of the face and neck. More specifically, the invention relates to a cosmetic method and an apparatus for improving the skin condition of the face and neck and improving sagging by suppressing differentiation of fat cells by thermal stimulation of the fat cells of the skin.

2. Description of the Related Art

The skin condition of the face and neck, especially sagging of the skin, is an important cosmetic concern. Nonetheless, no effective means of improvement has existed to date. Sagging of the skin of the abdomen, among various types of sagging of the skin, is believed to be caused by an increase in subcutaneous fat; that is, obesity. In contrast, sagging of the face and neck is believed to be caused by loss of tautness of the skin due to a decrease in the subcutaneous extracellular matrix rather than an increase in subcutaneous fat (Nihon Koshohin Gakkaishi [Journal of Japanese Cosmetic Science Society] Vol. 21, No. 3, pp. 190-196 (1997)). Methods for correcting the tautness of the skin using topical agents are believed to be effective in improving sagging of the skin of the face and neck in the past. An example of such an agent is an agent that promotes collagen production in fibroblasts. It is difficult, however, for these agents to reach the deeper levels of the skin. Another problem has been that such methods are merely palliative treatments.

Abnormal secretion of adipocytokines (adipokines), such as leptin, adiponectin, and the like, secreted from the adipose tissue or fat cells is known to occur with age and when the subcutaneous fat tissue is enlarged by obesity. For example, the secretion of adiponectin is suppressed in the enlarged subcutaneous fat tissue (FEBS Lett. 580: 2917-21 (2006)). On the other hand, adiponectin is known to inhibit the degradation of the extracellular matrix by inducing the expression of metalloproteinase inhibitor in macrophages (Circulation 109: 2046-49 (2004)). Skin fibroblasts were recently shown to produce extracellular matrix components such as collagen and the like in response to the humoral factors adiponectin, leptin, and the like produced by subcutaneous fat cells (U.S. Preliminary Patent Application 60/981,181 Specification (filed Oct. 19, 2007)). It is therefore suggested that dysfunction of adipose tissue associated with enlargement of adipose tissue, such as abnormal secretion of adipocytokines, causes sagging of the skin and lowers its viscoelasticity.

It is therefore necessary to develop a cosmetic method and apparatus for improving the condition of the skin by improving the adipose tissue dysfunction associated with enlargement of the adipose tissue such as abnormal secretion of adipocytokines. As a result of in-depth studies, the present inventors discovered that the differentiation of fat cells can be suppressed and the enlargement of the subcutaneous fat tissue can be prevented by applying thermal stimulation of a certain temperature. It is expected as a result that the skin condition will be improved, the viscoelasticity of the skin will be improved, and the cause of sagging of the skin will be eliminated by normalizing the abnormal secretion of adipocytokines.

### SUMMARY OF THE INVENTION

The present invention provides a cosmetic method for improving the skin condition of the face and/or neck. The cosmetic method for improving the skin condition of the present invention comprises a step for applying 60 to 90 minutes of thermal stimulation at 41 to 43° C. to subcutaneous fat of the face and/or neck.

The cosmetic method for improving the skin condition of the present invention may comprise a step for applying 60 minutes of thermal stimulation of 41.5 to 43° C. to the subcutaneous fat of the face and/or neck.

The skin condition may be sagging of the skin in the cosmetic method for improving skin condition of the present invention.

The present invention provides an apparatus for improving skin condition of the face and/or neck. The apparatus for improving skin condition of the present invention comprises a heater and applies 60 to 90 minutes of thermal stimulation at 41 to 43° C. to subcutaneous fat of the face and/or neck.

The apparatus for improving skin condition of the present invention may apply 60 minutes of thermal stimulation of 41.5 to 43° C. to the subcutaneous fat of the face and/or neck.

The heater of the apparatus for improving skin condition of the present invention may be a far-infrared lamp.

The apparatus for improving skin condition of the present invention may comprise a member for conducting heat energy of the heater to the skin of the face and/or neck.

The skin condition may be sagging of the skin in the apparatus for improving skin condition of the present invention.

### BRIEF DESCRIPTION OF THE DRAWINGS

FIG. 1 is a graph that shows the results of Oil Red staining of fat cell precursors cultured for 7 4 days after applying 60 minutes of thermal stimulation at 37° C., 39.5° C., 42° C., or 43° C. and inducing differentiation;

FIG. 2 is a graph that shows the results of Oil Red staining of fat cell precursors cultured for 7 4 days after applying 30 or 60 minutes of thermal stimulation at 43° C. and inducing differentiation;

FIG. 3 is a graph that shows the collagen production-promoting effect of adiponectin;

FIG. 4 is a graph that shows the hyaluronic acid production-promoting effect of adiponectin; and

FIG. 5 is a graph that shows the hyaluronic acid production-promoting effect of leptin.

### DESCRIPTION OF THE PREFERRED EMBODIMENTS

The temperature of the subcutaneous fat is raised to 39-43° C., preferably 41-43° C., more preferably 41.5-43° C., or 42-43° C., by thermal stimulation in this specification. The temperature when applying thermal stimulation in the present invention is preferably less than 43° C. This is because the skin keratinocytes and fibroblasts produce matrix metalloproteinase (MMP) and degradate the matrix at 43° C. and above (J. Invest. Dermatol. 123: 1012-1019, 2004). The effect of thermal stimulation in the present invention differs from that of conventional thermal stimulation such as a bath or hot gel application. Specifically, conventional thermal stimulation can be expected to combust or degrade the fat by accelerating metabolic activity by promoting blood flow. In contrast to this, the thermal stimulation in the present invention has the effect of promoting matrix production by suppressing differentiation of subcutaneous fat cells. The present invention is therefore clearly distinct from conventional methods of improving the skin condition, especially sagging of the skin, that are only palliative remedies insofar as it makes it possible to improve the skin condition, especially sagging of the skin, by targeting the mechanism that creates sagging of the skin due to obesity.

The duration of heat by thermal stimulation in the present specification is 30 minutes or more, preferably 30-120 minutes, more preferably 60-90 minutes, and most preferably 60 minutes. This thermal stimulation is sometimes a stepwise treatment that combines two or more different temperatures and times. A single thermal stimulation treatment may be conducted continuously or intermittently by inserting rest periods. This thermal stimulation treatment may be conducted once, twice or more times a day, every day, or at intervals.

The thermal stimulation of the present invention is sometimes conducted, for example, by heating by movement of the muscles, by heating the target site by nerve stimulation, or by other such methods of generating heat within the body. Thermal stimulation is also sometimes conducted, for example, by heating deep regions of the skin using a heater.

The heater in the apparatus for improving skin condition of the present invention may be a far infrared lamp. The heater may be a heater that utilizes a chemical exothermal reaction or a heater that uses electrothermic equipment or another heater that uses electrical energy. The heater may transfer the heat energy to the skin of the face and/or neck by radiant heat, but the heat energy of the heater may also be transferred by a member that conducts the heat energy to the skin of the face and/or neck.

The apparatus for improving skin condition of the present invention may comprise a member for avoiding or mitigating secondary effects such as drying of the skin or swelling of the skin due to perspiration and other skin moisture during the time that thermal stimulation is being applied; and a sheet, cushion, or pillow to maintain the body position so that the subject receives the thermal stimulation comfortably. Other apparatus may also be provided to permit treatments that stimulate other senses, such as music therapy, light therapy, aromatherapy, massage, and the like, at the same time as the subject is receiving thermal stimulation.

As is explained in Example 1 below, the differentiation of fat cell precursors into fat cells is suppressed by applying thermal stimulation to fat cell precursors. As a result, enlargement of the fat cells in the subcutaneous fat is suppressed. As described above, enlargement of the fat cells is known to lower the blood concentration of adiponectin secreted by the fat cells.

On the other hand, as will be explained in Examples 2 to 4, fibroblasts in the skin produce collagen and hyaluronic acid, which are extracellular matrix components, in response to adiponectin and leptin, which are humoral factors produced by the subcutaneous fat cells. In other words, the adiponectin produced by the fat cells promotes the production of a matrix such as collagen, hyaluronic acid, and the like by the fibroblasts in the dermis.

When enlargement of the fat cells is suppressed, therefore, lowering of the blood concentration of adiponectin is prevented and fibroblastic production of collagen and hyaluronic acid by the adiponectin is promoted. As a result, the tautness of the skin of the face and/or neck is improved and sagging is improved. The thermal stimulation of the present invention provides an effect of improving the dysfunction of the adipose tissue, such as abnormal secretion of adiponectin, associated with enlargement of the adipose tissue and improves the skin condition, especially sagging of the skin, of the face and/or neck by specifically suppressing the differentiation of the fat cells in the subcutaneous fat of the face and/or neck.

It was discovered based on the results of Examples 2 to 4 that the subcutaneous fat cells directly stimulate the production of the extracellular matrix in the skin tissue. This discovery led to a method of improving the skin condition, especially loss of viscoelasticity of the skin, that comprises a step for administering an adipokine. The step for administering the adipokine according to the present method of improving the skin condition may be a step for enhancing adipokine production in the subcutaneous fat cells. The step for enhancing adipokine production in the subcutaneous fat cells may be achieved by mechanical stimulation of the subcutaneous fat cells. The step for enhancing adipokine production in the subcutaneous fat cells may be achieved by chemical stimulation of the subcutaneous fat cells.

The discovery led to a method of using an adipokine in order to improve the skin condition, especially loss of viscoelasticity of the skin. The discovery led to a method of evaluating a skin condition improvement technique comprising a step for measuring the amount of adipokine produced in the subcutaneous fat cells. The step for measuring the amount of adipokine produced in the subcutaneous fat cells in the method of evaluating a skin condition improvement technique may comprise measuring the amount of adipokine mRNA present in the subcutaneous fat cells. The step for measuring the amount of adipokine produced in the subcutaneous fat cells may comprise measuring the amount of adipokine protein present in the subcutaneous fat cells. The step for measuring the amount of adipokine produced in the subcutaneous fat cells may comprise measuring the amount of adipokine protein present in the subcutaneous extracellular matrix.

In the method for improving skin condition, method for using an adipokine to improve the skin condition and method for evaluating a skin condition improvement technique, the adipokine may be at least one protein selected from the group consisting of leptin, adiponectin, HB-HGF, IL-6, resistin and TNF-alpha. The adipokine in the method of the present invention may be at least one protein selected from the group consisting of leptin and adipokine. The adipokine in the method may be leptin. The adipokine in the method may be adiponectin. The adipokine in the method of improving the skin condition may be adiponectin, and the chemical stimulation may be a PPARγ agonist.

The discovery led to a pharmaceutical composition to improve the skin condition, especially loss of viscoelasticity of the skin, that contains an adipokine. The adipokine in the pharmaceutical composition may be at least one protein selected from the group consisting of leptin, adiponectin, HG-HGF, IL-6, resistin and TNF-alpha. The adipokine in the pharmaceutical composition may be at least one protein selected from the group consisting of leptin and adiponectin. The adipokine in the pharmaceutical composition may be leptin. The adipokine in the pharmaceutical composition may be adiponectin.

The discovery led to a kit for evaluating a skin condition improvement technique that includes a polynucleotide probe or polynucleotide primer to detect adipokine mRNA, and instructions that describe a method of evaluating a skin condition improvement technique which includes the measurement of the amount of adipokine mRNA present in the subcutaneous fat cells. The measurement of the amount of adipokine mRNA is not restricted to a specific method, but it is preferable to use real-time PCR. Northern blotting or solid-phase hybridization may be used in the measurement of the amount of mRNA in the present invention. Solid-phase hybridization includes, but is not limited to, multi-array hybridization whereby mRNA or a labeled nucleic acid derived from mRNA is hybridized to an array of probes for different genes arranged two-dimensionally; and bead hybridization whereby mRNA or a labeled nucleic acid derived from mRNA is hybridized to beads on which probes of different genes have been immobilized. The nucleotide sequence that codes the adipokine of the present invention can be obtained easily through Internet sites known to those skilled in the art such as the DNA Data Bank of Japan (DDBJ) and Entrez provided by the United States NCBI.

The discovery led to a kit for evaluating a skin condition improvement technique that includes a specific binding partner to detect an adipokine protein, and instructions that describe a method of evaluating a skin condition improvement technique which includes the measurement of the amount of adipokine protein present in the subcutaneous extracellular matrix. The specific binding partner to detect an adipokine protein in the kit for evaluating a skin condition improvement technique may be an antibody. The adipokine in the kit for evaluating a skin condition improvement technique may be at least one protein selected from the group consisting of leptin, adiponectin, HB-HGF, IL-6, resistin and TNF-alpha. The adipokine in the kit for evaluating a skin condition improvement technique may be at least one protein selected from the group consisting of leptin and adiponectin. The adipokine in the kit for evaluating a skin condition improvement technique may be leptin. The adipokine in the kit for evaluating a skin condition improvement technique may be adiponectin. The specific binding partner to detect the adipokine protein includes, but is not limited to, a specific antibody that binds to the adipokine and a protein that is comprised of all or part of the adipokine receptor protein. Adipokine protein and specific binding partners to detect adipokines are available commercially. For example, human leptin, anti-human leptin antibody, human adiponectin, and anti-human adiponectin antibody manufactured by Alpha Diagnostic International, Inc. are all available from Nacalai Tesque.

The examples below are intended to illustrate the present invention and not to limit the scope of the present invention. The scope of the present invention should be interpreted on the basis of the language of the claims attached to this specification.

### Example 1

### 1. Study of the Effect of Thermal Stimulation on the Differentiation of fat Precursor Cell

### 1-1. Materials and Methods

Mouse 3T3-L1 cells were used as fat precursor cells. The cells were inoculated at 1.5×10⁴ cells/well in 24-well plates. The 24 wells were then divided into three groups of 8 wells, and each group was subjected to thermal stimulation. The thermal stimulation conditions were 60 minutes at a temperature of 37° C., 39.5° C., 42° C. or 43° C. for each group. After thermal stimulation, insulin, dexamethasone and isobutylmethylxanthine (IBMX) were added to each well to the final concentrations of 0.2, 0.3 and 200 μM, respectively, and the cells were cultured for 7 days at 37° C. The LDH activity in the culture supernatant was measured after two days of culture. After 7 4 days of culture, the triglyceride in the cells that had differentiated into fat cells was stained by Oil Red. Specifically, after removing the culture medium and washing with PBS, the cells were fixed with 10% neutral formalin solution and rinsed with distilled water, then stained by 0.35% Oil Red solution. Quantitative measurement was conducted by rinsing with distilled water after staining, extracting the Oil Red by isopropanol, and measuring the 540 nm absorbance. Experiments were also carried out by setting the thermal stimulation conditions of each group for 30 or 60 minutes at 43° C.

### 1-2. Results

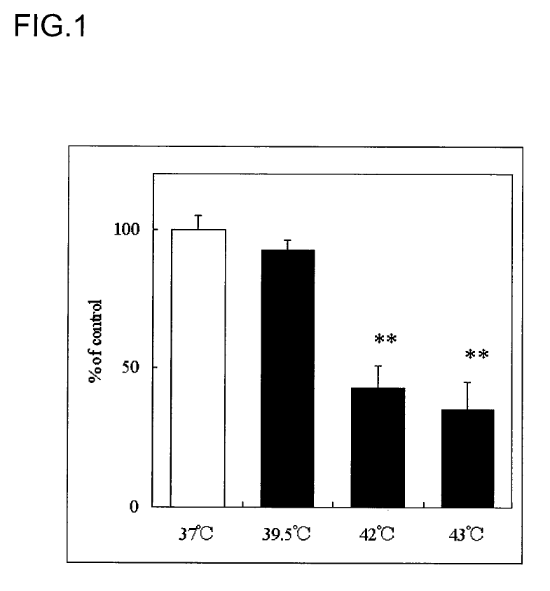

FIG. 1 is a graph that indicates quantitatively the degree of differentiation of fat cell precursors into fat cells by Oil Red staining when cultured for 7 4 days after conducting 60 minutes of thermal stimulation at 37° C., 39.5° C., 42° C. or 43° C. and inducing differentiation by insulin and the like. There was a decrease to about 50% in the 42° C. group and a decrease to about 35% in the 43° C. group in comparison to the 37° C. group. A risk of p<0.0021 at 42° C. and p<0.000106 at 43° C. was calculated with Dunnett's test in order to test the difference from the results at 37° C., and the degree of differentiation of the fat cells precursors into fat cells in the 42° C. and 43° C. groups was statistically significant in comparison to the 37° C. group. The results demonstrated that thermal stimulation of the fat precursor cells suppresses the differentiation of the fat cell precursors into fat cells.

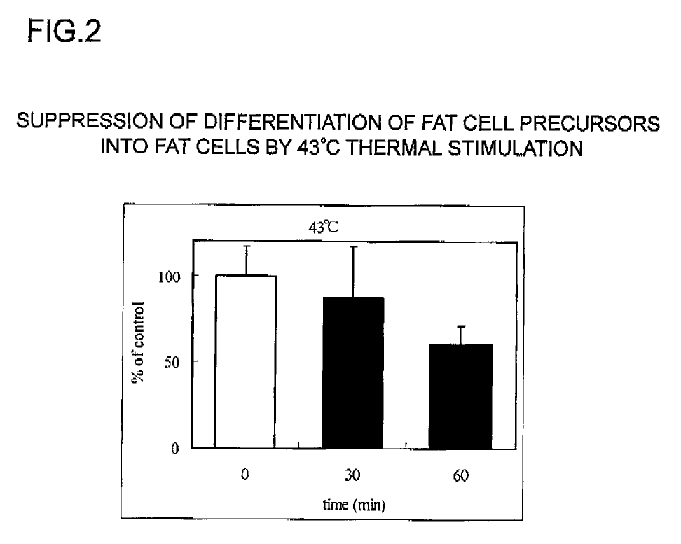

FIG. 2 is a bar graph that shows the Oil Red staining results of fat cell precursors cultured for 7 4 days after conducting 30 or 60 minutes of thermal stimulation at 43° C. and inducing differentiation by insulin and the like. As in FIG. 1, FIG. 2 indicates the relative values, 100% being the result of the group not subjected to thermal stimulation (control). The absorbance decreased about 10% in the 30-minute group and about 40% in the 60-minute group in comparison to the 37° C. group. Based on the results of measurement of the LDH activity (not shown), the cells were not damaged by thermal stimulation. The result demonstrated that thermal stimulation of the fat cell precursors suppresses the differentiation of the fat cell precursors into fat cells.

### Example 2

### 2. Study of the Effect of Humoral Factors Produced by Fat Cells on Extracellular Matrix Production by Skin Fibroblasts

### 2-1. Materials and Methods

Human skin fibroblasts (Sanko Junyaku) cultured by D-MEM supplemented with 10% FBS were inoculated on 24-well plates, and the medium was replaced with D-MEM supplemented with 0.5% FBS after five hours of culture. After another seven hours, various concentrations of adiponectin (Nacalai Tesque) or leptin (Sigma) were added, and the medium was collected after a certain period of time. The amounts of collagen and hyaluronic acid in the collected medium were measured by an ELISA kit (collagen: Takara Bio; hyaluronic acid: Seikagaku).

### 2. Results

### 2-2. Collagen Production-Promoting Effect of Adiponectin

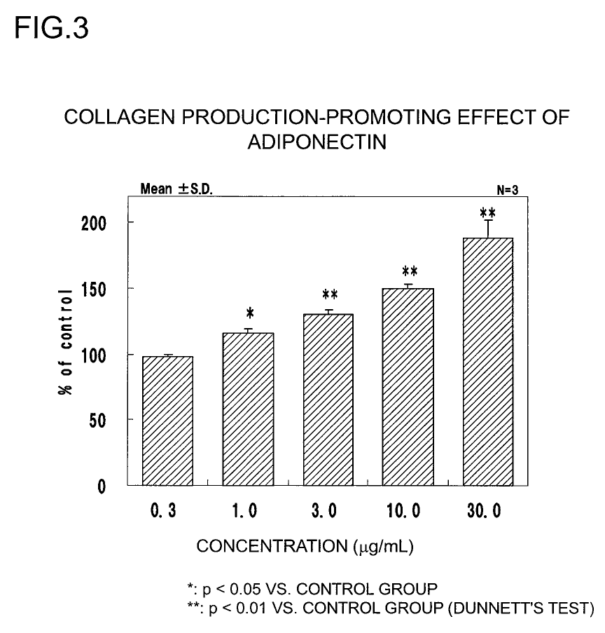

FIG. 3 indicates the results of experiments performed to examine the collagen production-promoting effect of adiponectin. The amount of collagen produced in human skin fibroblasts in the presence of 0.3, 1.0, 3.0, 10.0, and 30.0 μg/mL of adiponectin is represented by the relative value, 100% being the amount of collagen produced from the cells in the absence of adiponectin. The amount of collagen produced in the presence of adiponectin was 100% or more in all cases, and it was demonstrated that the amount of collagen production increases in correspondence to the adiponectin concentration.

### Example 3

### 2-3. Hyaluronic Acid Production-Promoting Effect of Adiponectin

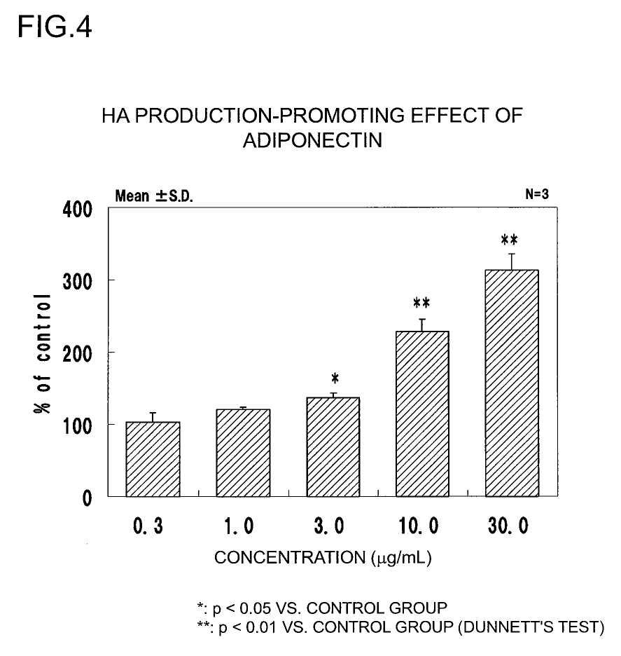

FIG. 4 illustrates the results of experiments performed to study the hyaluronic acid production-promoting effect of adiponectin. The amount of hyaluronic acid produced from human skin fibroblasts in the presence of 0.3, 1.0, 3.0, 10.0, and 30.0 μg/mL of adiponectin is represented by the relative value, 100% being the amount of hyaluronic acid produced in the cells in the absence of adiponectin. The amount of collagen produced in the presence of adiponectin was 100% or more in all cases, and demonstrated that the amount of hyaluronic acid produced increases in correspondence to the adiponectin concentration.

### Example 4

### 2-4. Hyaluronic Acid Production-Promoting Effect of Leptin

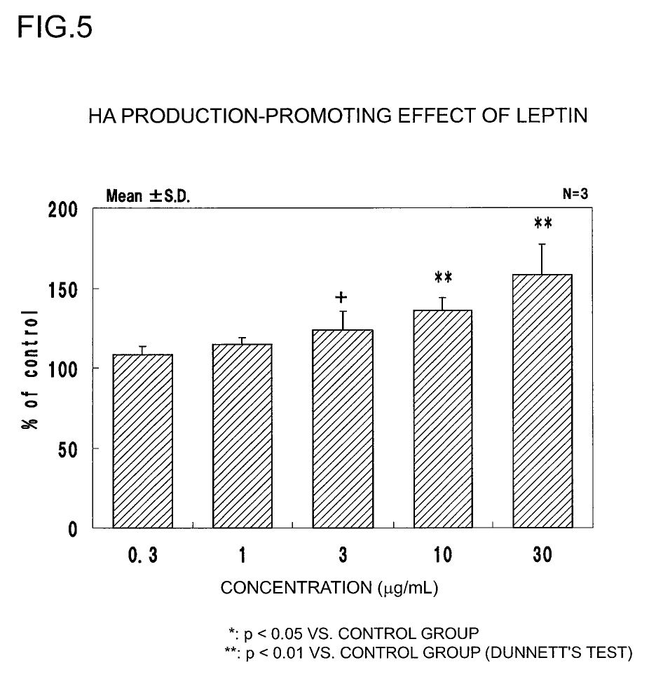

FIG. 5 illustrates the results of experiments performed to examine the hyaluronic acid production-promoting effect of leptin. The amount of hyaluronic acid produced in human skin fibroblasts in the presence of 0.3, 1, 3, 10, and 30 μg/mL of leptin is represented by the relative value, 100% being the amount of hyaluronic acid produced from the cells in the absence of leptin. The amount of collagen produced in the presence of leptin was 100% or more in all cases, and demonstrated that the amount of hyaluronic acid produced increases in correspondence to the leptin concentration.

What is claimed is:

1. A cosmetic method for improving a skin condition of the face and/or neck comprising a step for providing 60 to 90 minutes of thermal stimulation at 41 to 43° C. to subcutaneous fat of the face and/or neck.

2. The cosmetic method of claim 1 comprising a step for providing 60 minutes of thermal stimulation at 41.5 to 43° C. to the subcutaneous fat of the face and/or neck.

3. The cosmetic method of claim 1, wherein the skin condition is sagging of the skin.

4. The cosmetic method of claim 2, wherein the skin condition is sagging of the skin.

5. An apparatus for improving a skin condition of the face and/or neck comprising a heater and providing 60 to 90 minutes of thermal stimulation at 41 to 43° C. to subcutaneous fat of the face and/or neck.

6. The apparatus for improving the skin condition of claim 5 providing 60 minutes of thermal stimulation at 41.5 to 43° C. to the subcutaneous fat of the face and/or neck.

7. The apparatus for improving the skin condition of claim 5, wherein the heater is a far-infrared lamp.

8. The apparatus for improving the skin condition of claim 6, wherein the heater is a far-infrared lamp.

9. The apparatus for improving the skin condition of claim 5 comprising a member for conducting heat energy of the heater to the skin of the face and/or neck.

10. The apparatus for improving the skin condition of claim 6 comprising a member for conducting heat energy of the heater to the skin of the face and/or neck.

11. The apparatus for improving the skin condition of claim 5, wherein the skin condition is sagging of the skin.

12. The apparatus for improving the skin condition of claim 6, wherein the skin condition is sagging of the skin.

13. The apparatus for improving the skin condition of claim 7, wherein the skin condition is sagging of the skin.

14. The apparatus for improving the skin condition of claim 8, wherein the skin condition is sagging of the skin.

15. The apparatus for improving the skin condition of claim 9 wherein the skin condition is sagging of the skin.

16. The apparatus for improving the skin condition of claim 10 wherein the skin condition is sagging of the skin.
